(19) United States
(12) Patent Application Publication
(10) Pub. No.: US 2022/0143260 A1
(43) Pub. Date: May 12, 2022

(54) ULTRA-VIOLET LED DEVICE FOR DISINFECTING ROOM AIR

(71) Applicant: Dynamics Energy, LLC, Eagle, ID (US)

(72) Inventors: Richard C. Newbold, III, Philadelphia, PA (US); Robert W. Abbott, Boise, ID (US)

(21) Appl. No.: 17/523,178

(22) Filed: Nov. 10, 2021

Related U.S. Application Data
(60) Provisional application No. 63/112,779, filed on Nov. 12, 2020.

(51) Int. Cl. A61L 9/20

(52) U.S. Cl. CPC A61L 9/20; A61L 2209/12

(57) ABSTRACT

An air disinfecting system for use in an enclosed space is disclosed. The system includes an air chamber having an inlet end and an outlet end, a check valve at the inlet end, an LED array comprising at least one UVC LED light within the air chamber, a fan to move air through the air chamber, and a controller to control operation of the at least one UVC LED light and the one fan.

### CROSS-REFERENCE TO RELATED APPLICATIONS

This application, under 35 U.S.C. § 119, claims the benefit of U.S. Provisional Patent Application Ser. No. 63/112,779 filed on Nov. 12, 2020, and entitled “ULTRA-VIOLET LED DEVICE FOR DISINFECTING ROOM AIR,” the contents of which are hereby incorporated by reference herein.

### FIELD OF THE DISCLOSURE

This disclosure relates generally to apparatus and methods for enhancing personal and societal protection from airborne pathogens via air systems. More particularly, this disclosure relates to a building or room sized capacity system that exploits ultra-violet C wavelength (UVC) radiation for its germicidal and disinfectant effects.

### BACKGROUND

Airborne pathogens, such as the COVID-19, SARS, and the like viruses, can cause global pandemics and wreak havoc with societal and economic norms. As airborne pathogens transmit and travel through the air, protective equipment, such as face masks, are often prescribed to mitigate pathogen transmission. However, existing face masks and the like, typically operate to block transmission of airborne pathogens and do not otherwise disinfect inhaled or exhaled air. Blocking transmission is rarely 100% effective and transmission of pathogens can still occur. Furthermore, wearing of facemasks is often inconvenient or impossible in some environments and conditions. Accordingly, there is a need for systems and methods to disinfect air in an entire room, building, or other enclosed space.

In highly infectious environments, such as hospitals or other health care facilities, physicians, nurses, first responders, and other health care workers, can be exposed to unsafe levels of airborne pathogens. In such environments the need for effective air-filtration equipment is enhanced. Other needs for effective filtration equipment, and drawbacks of existing solutions, also exist.

### SUMMARY

Accordingly, disclosed embodiments address the above, and other, needs and drawbacks of existing systems and methods. Disclosed embodiments include an air disinfecting system for use in an enclosed space, the system including a first air chamber having a first inlet end and a first outlet end. The system includes at least one check valve at the first inlet end, at least one LED array having at least one UVC LED light within the first air chamber, at least one fan to move air through the first air chamber, and a controller to control at least operation of the at least one UVC LED light and the at least one fan.

Further disclosed embodiments of the system include a second air chamber having a second inlet end and a second outlet end, a U-bend air chamber connected between the first outlet end of the first air chamber and the second inlet end of the second air chamber, and at least one LED array having at least one UVC LED light within the second air chamber.

Further disclosed embodiments include at least one filter at the first inlet end. In still further disclosed embodiments the at least one UVC LED light emits light substantially in the wavelengths from 200 nm to 280 nm.

In some disclosed embodiments the first air chamber is semi-circular in cross-section. In other disclosed embodiments the first air chamber is circular in cross-section.

Further disclosed embodiments include a reflective surface on an interior surface of the first air chamber. In still further disclosed embodiments the reflective surface comprises a reflective polytetrafluoroethylene (PTFE) surface. In other disclosed embodiments, the reflective surface comprises a sandblasted aluminum surface.

Further disclosed embodiments include a third air chamber having a third inlet end and a third outlet end, a U-bend air chamber connected between the second outlet end of the second air chamber and the third inlet end of the third air chamber, and at least one LED array comprising at least one UVC LED light within the third air chamber.

Other embodiments are also disclosed.

### BRIEF DESCRIPTION OF THE DRAWINGS

FIG. 1 is schematic illustration of a system for air disinfecting in accordance with disclosed embodiments.

FIG. 2 is a schematic cross-sectional view of an air chamber in accordance with disclosed embodiments.

FIGS. 3A-3B are schematic cross-sectional views of air chambers in accordance with disclosed embodiments.

While the disclosure is susceptible to various modifications and alternative forms, specific embodiments have been shown by way of example in the drawings and will be described in detail herein. However, it should be understood that the disclosure is not intended to be limited to the particular forms disclosed. Rather, the intention is to cover all modifications, equivalents and alternatives falling within the spirit and scope of the invention as defined by the appended claims.

### DETAILED DESCRIPTION

Disclosed exemplary embodiments include apparatus for continuously disinfecting air in rooms, buildings, and other enclosed spaces by inactivating airborne pathogens and infected aerosols with high intensity ultra-violet C-band (UVC) light in a device designed to maximize microbial residence time, UVC irradiance, and UVC fluence (dosage). As used herein, the terms “room,” “building,” and “enclosed space” are understood to have their conventional meaning of a generally human-occupiable space with a generally controllable climate and include, but are not limited to, homes, office buildings, hospitals, schools, laboratories, theaters, restaurants, and subdivided spaces within the same, such as bedrooms, bathrooms, kitchens, offices, retail stores, dining rooms, computer rooms, classrooms, gyms, and the like. Enclosed spaces may also include vehicles with generally controllable climates, such as, but not limited to, recreational vehicles, airplanes, cars, trucks, busses, ships, boats, and the like. Persons of ordinary skill in the art having the benefit of this disclosure would understand that the capacity and size of the disinfecting apparatus can be scaled and adapted in accordance with the size and configuration of a particular room, building, or enclosed space.

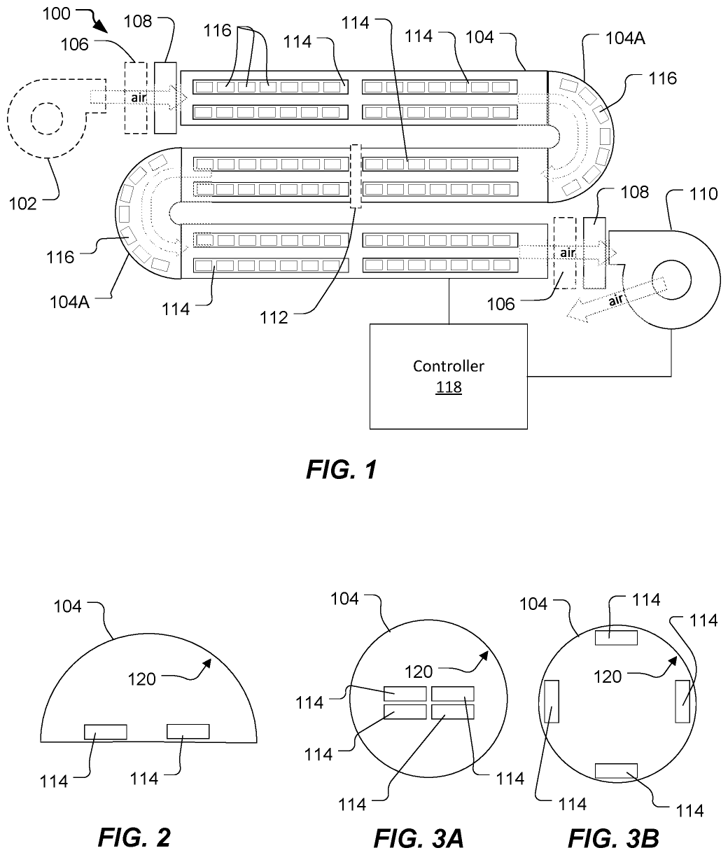

FIG. 1 is schematic illustration of a system 100 for air disinfecting in accordance with disclosed embodiments. As shown, system 100 may be electrically powered (e.g., wired into the building's electrical system, with stand-alone power, battery powered, or the like) and may be configured as a continuous, cylindrical or hemi-cylindrical air chamber of variable length and volume with one or more internal fans to collect, hold (for a set interval), purify, and discharge room air in a repeating cycle the periodicity of which can be determined by the end-user input to a controller which may include adjustable timers or the like communicating with the fan(s).

As shown in FIG. 1, some embodiments may include an optional fan, blower, or other air circulation device, such as fan 102, to direct room air into air chamber 104 as indicated by the arrows. In some embodiments, the blower or fan 110 that ultimately discharges disinfected air back into the room may be of a capacity and size that fan 102 is not required, or a single blower fan 102 may be placed at the inlet of the system 100 in some embodiments. In other embodiments, in particular when air chamber 104 is of an extended length or diameter, one or more inline fans 112 may optionally be provided to facilitate air flow through air chamber 104.

Embodiments of system 100 may include an optional dust or other particulate filter 106 which may be cleanable or replaceable. As also indicated, embodiments of system 100 include a one-way check valve 108 to prevent or limit backflow of air into the room. Similar filtration 106 and check valves 108 may be provided at the inlet and the outlet of the system 100 in some embodiments.

As also shown schematically in FIG. 1, a number of LED arrays 114 comprising a number of UVC LED lights 116 are provided within air chamber 104. While shown in generally rectangular arrays 114 in FIG. 1, other shapes and configurations may also be used. For example, one or more U-bend air chamber 104A may be included in system 100 and UVC LED lights 116 may be spaced curvilinearly through the U-bend chamber 104A as shown. In some embodiments, UVC LED lights 116 emit light in substantially 275 nm wavelengths. Additionally, as persons of ordinary skill in the art having the benefit of this disclosure would comprehend, more or fewer U-bend air chambers 104A may be included to extend, or lessen, the air flow passage length as desired.

As shown schematically in FIG. 1, controller 118 is provided in some embodiments to control the on/off state of the LED arrays 114, the fans 102, 110, 112, and any other system 100 components requiring control (e.g., check valves 108, timers to alert replacement times for filters 106, and the like).

FIG. 2 is a schematic cross-sectional view of an air chamber 104 in accordance with disclosed embodiments. As shown, in some embodiments air chamber 104 may be substantially hemicylindrical, or semi-circular in cross section, to facilitate reflection of UVC light from the LED arrays 114. In some embodiments a reflective surface 120 may be provided on the interior surface of air chamber 104. Embodiments of reflective surface 120 may include a reflective polytetrafluoroethylene (PTFE) surface, a sandblasted aluminum surface, or the like.

FIGS. 3A-3B are schematic cross-sectional views of air chambers 104 in accordance with disclosed embodiments. As indicated in FIG. 3A-3B embodiments of air chamber 104 may have a circular cross-section (i.e., a cylindrical pipe or the like). As also indicated LED arrays 114 may be configured in a generally central location in the air chamber 104 (e.g., FIG. 3A), distributed about the inner surface (e.g., FIG. 3B), or in other configurations to facilitate reflection of the UVC light throughout the air chamber 104.

In some embodiments, the programming of the air-flow cycle (e.g., with controller 118) allows highly infected air to reside longer in the air chamber 104 and dramatically increasing the antimicrobial efficacy of the UVC light. In other embodiments, the system 100 can be operated continuously at lower or higher fan speeds (e.g., fans 102, 110, 112) to exchange room air more frequently if lower levels of air purification/sterilization are desired. The device exploits the proven germicidal efficacy of ultra-violet C wavelength

(UVC) radiation supplied by LED lights 116 is augmented by lining the interior of the air chamber 104 with high-reflectance material 120. The choice of cylindrical or hemi-cylindrical shape for the air chamber 104 is deliberate to increase the Lambertian reflectance of photons and thereby increase the dosage of UVC delivered to airborne pathogens. A replaceable dust filter 106 of medium air resistance may be positioned at the intake opening. The system 100 may be self-contained and may be powered by rechargeable batteries or other power supply. System 100 can be configured for permanent installation on ceilings (standard ceiling or drop-down), walls, or as a free-standing, floor unit. The system 100 is scalable by extending the length of the continuous purification air chamber 104 and/or augmenting the volume capacity and refabricating in an appropriate housing or cabinet.

For example, in an embodiment intended for ceiling tile placement, the system 100 (measuring 2×2 feet×6 inches) may have an internal half-cylinder radius of 4 inches and a total internal volume of 1,808.6 cubic inches. With the programmable controller 118, fans (e.g., 102, 110, 112) set on 4-second fill and 6 second static air residence time, 6 cycles are completed every minute and slightly over 1.5 complete room air exchanges/purifications are accomplished every 24 hours.

As an extended single-chamber 104 configuration with variable residence (air holding) time set on maximum, the system 100 can provide “single-pass” protection from virtually all airborne pathogens. Many other embodiments can be incorporated into existing air conduits, HVAC systems, or other filtration devices, programmed to optimize air residence time and maximize microbial sterilization.

Air is purified by UVC-wavelength LED lights 116 housed in one continuous chamber 104 of variable length and diameter lined with ultra-UVC reflecting material; unidirectional air check valves 108 in the UVC-reflecting surface 120 in air chamber 104 disinfects all air. To maximize reflectance and therefore fluence (dosage), a cylindrical or hemi-cylindrical shape is preferred; however, the shape of the chamber 104 can also be rectangular, or other shapes.

Air enters, transits, and exits the air chamber 104 through operation of fan(s) 102, 110 located at one or both ends of the chamber. The air chamber 104 is relatively unobstructed and designed for minimal airway resistance. Pathogens in the air are subjected to extremely high intensity UVC light in the germicidal wavelength range of 275 nm of sufficient energy to inactivate virtually all airborne pathogenic microbes (bacteria, viruses, fungi and spores) and achieve up to 99.999% sterilization with all known human pathogenic viruses. No pathogenic organisms are resistant to UVC's antimicrobial impact as its mechanism of action is to disrupt DNA and/or RNA reproduction, repair, and translation.

Although various embodiments have been shown and described, the present disclosure is not so limited and will be understood to include all such modifications and variations are would be apparent to one skilled in the art.

What is claimed is:

1. An air disinfecting system for use in an enclosed space, the system comprising:
a first air chamber comprising a first inlet end and a first outlet end;
at least one check valve at the first inlet end;
at least one LED array comprising at least one UVC LED light within the first air chamber;
at least one fan to move air through the first air chamber; and
a controller to control at least operation of the at least one UVC LED light and the at least one fan.

2. The air disinfecting system for use in an enclosed space of claim 1 further comprising:
a second air chamber comprising a second inlet end and a second outlet end;
a U-bend air chamber connected between the first outlet end of the first air chamber and the second inlet end of the second air chamber; and
at least one LED array comprising at least one UVC LED light within the second air chamber.

3. The air disinfecting system for use in an enclosed space of claim 1 further comprising:
at least one filter at the first inlet end.

4. The air disinfecting system for use in an enclosed space of claim 1 wherein the at least one UVC LED light emits light substantially in the wavelengths from 200 nm to 280 nm.

5. The air disinfecting system for use in an enclosed space of claim 1 wherein the first air chamber is semi-circular in cross-section.

6. The air disinfecting system for use in an enclosed space of claim 1 wherein the first air chamber is circular in cross-section.

7. The air disinfecting system for use in an enclosed space of claim 1 further comprising:
a reflective surface on an interior surface of the first air chamber.

8. The air disinfecting system for use in an enclosed space of claim 7 wherein the reflective surface comprises a reflective polytetrafluoroethylene (PTFE) surface.

9. The air disinfecting system for use in an enclosed space of claim 7 wherein the reflective surface comprises a sandblasted aluminum surface.

10. The air disinfecting system for use in an enclosed space of claim 2 further comprising:
a third air chamber comprising a third inlet end and a third outlet end;
a U-bend air chamber connected between the second outlet end of the second air chamber and the third inlet end of the third air chamber; and
at least one LED array comprising at least one UVC LED light within the third air chamber.
